(19) United States
(12) Patent Application Publication
(10) Pub. No.: US 2023/0144677 A1
(43) Pub. Date: May 11, 2023

(54) USER INTERFACE WITH INTEGRATED SENSORS

(71) Applicant: ResMed Sensor Technologies Limited, Dublin (IE)

(72) Inventors: Stephen McMahon, Dublin (IE); Redmond Shouldice, Dublin (IE)

(21) Appl. No.: 17/915,060

(22) PCT Filed: Mar. 27, 2021
(86) PCT No.: PCT/IB2021/052557

§ 371 Date: Sep. 27, 2022

Related U.S. Application Data
(60) Provisional application No. 63/001,273, filed on Mar. 28, 2020.

(51) Int. Cl. A61M 16/06; A61M 16/08; A61M 16/00

(52) U.S. Cl. CPC A61M 16/0622; A61M 16/0003; A61M 16/022; A61M 16/0683; A61M 16/0816

(57) ABSTRACT

A user interface of a respiratory therapy system includes a strap assembly, a frame, a connector, and a sensor. The strap assembly is positioned about a head of a user when the user wears the user interface. The frame is physically and electrically connected to the strap assembly, and defines an aperture. The connector has a first end portion and second end portion. The first end portion of the connector can be positioned within the aperture of the frame such that the connector is physically and electrically connected to the frame. The sensor is coupled to the strap assembly or the frame such that the sensor abuts a target area of the user when the user wears the user interface.

### CROSS-REFERENCE TO RELATED APPLICATIONS

This application claims the benefit of, and priority to, U.S. Provisional Pat. Application No. 63/001,273 filed on Mar. 28, 2020, which is hereby incorporated by reference herein in its entirety.

### TECHNICAL FIELD

The present disclosure relates generally to systems and methods for analyzing data related to a user using a respiratory therapy system, and more particularly, to systems and methods for positioning sensors in a user interface worn by a user during use of the respiratory therapy system.

### BACKGROUND

Many individuals suffer from sleep-related disorders, such as insomnia (e.g., difficulty initiating sleep, frequent or prolonged awakenings after initially falling asleep, and an early awakening with an inability to return to sleep), periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), hypertension, diabetes, stroke, etc. Many of these sleep related disorders can be treated or managed more effectively if certain data about the is received and analyzed. However, it can be difficult to utilize sensors in a manner that is able to capture the desired data without interrupting the user’s sleep or treatment. Thus, it would be advantageous to locate sensors in the user interface that the user wears during sleep and treatment. The present disclosure is directed to solving these and other problems.

### SUMMARY

According to some implementations of the present disclosure, a user interface of a respiratory therapy system comprises a strap assembly configured to be positioned generally about at least a portion of a head of a user when the user interface is worn by the user; a frame physically and electrically connected to the strap assembly, the frame defining an aperture; a connector having a first portion and a second portion, the first portion being configured to be at least partially positioned within the aperture of the frame such that the connector is physically and electrically connected to the frame; and a sensor coupled to the strap assembly or the frame such that the sensor abuts a target area of the user when the user interface is worn by the user.

According to some implementations of the present disclosure, a respiratory therapy device comprises a housing defining an inlet and an outlet; a blower motor positioned within the housing in fluid communication with the inlet and the outlet; a memory device storing machine readable instructions; and a control system including one or more processors configured to execute the machine-readable instructions to cause the blower motor to flow pressurized air out of the outlet, wherein the respiratory therapy device does not include a pressure sensor positioned within the housing and wherein the respiratory therapy device does not include a flow rate sensor positioned within the housing.

According to some implementations of the present disclosure, a user interface of a respiratory therapy system comprises a strap assembly configured to be positioned generally about at least a portion of a head of a user when the user interface is worn by the user; a frame physically and electrically connected to the strap assembly, the frame defining an aperture; a cushion coupled to the frame and positioned between the frame and the strap assembly, a connector having a first portion and a second portion, the first portion being configured to be at least partially positioned within the aperture of the frame such that the connector is physically and electrically connected to the frame; and a non-contact sensor positioned within the frame or within the cushion area of the user.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a respiratory therapy system, according to some implementations of the present disclosure;

FIG. 2 is a perspective view of the respiratory therapy system of FIG. 1, a user of the respiratory therapy system, and a bed partner of the user, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5A is a perspective view of a first implementation of a user interface of the respiratory therapy system of FIG. 1, according to some implementations of the present disclosure;

FIG. 5B is a perspective exploded view of the user interface of FIG. 3A, according to some implementations of the present disclosure;

FIG. 6A is a perspective view of the alignment of electrical contacts of a connector and a frame of the user interface of FIG. 5A, according to some implementations of the present disclosure;

FIG. 6B is a magnified view of the electrical contacts of the frame of the user interface of FIG. 5A, according to some implementations of the present disclosure;

FIG. 6C is a cross-sectional view of the electrical connection between the connector and the frame of the user interface of FIG. 5A prior to the connector being inserted into the frame, according to some implementations of the present disclosure;

FIG. 6D is a cross-sectional view of the electrical connection between the connector and the frame of the user interface of FIG. 5A after the connector is inserted into the frame, according to some implementations of the present disclosure;

FIG. 7 is a perspective view of the electrical connection between the frame and a strap of the user interface of FIG. 5A, according to some implementations of the present disclosure; and

FIG. 8 is a perspective view of a user wearing the user interface of FIG. 5A, according to some implementations of the present disclosure.

FIG. 9A is a perspective view of a second implementation of a user interface of the respiratory therapy system of FIG. 1, according to some implementations of the present disclosure.

FIG. 9B is an exploded view of the user interface of FIG. 9A, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory-related disorders. Examples of sleep-related and/or respiratory-related disorders include Periodic Limb Movement Disorder (PLMD), Restless Leg Syndrome (RLS), Sleep-Disordered Breathing (SDB), Obstructive Sleep Apnea (OSA), Central Sleep Apnea (CSA), other types of apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), chest wall disorders, and rapid eye movement (REM) behavior disorder, also referred to as RBD.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall.

Central Sleep Apnea (CSA) is another form of SDB that results when the brain temporarily stops sending signals to the muscles that control breathing. More generally, an apnea generally refers to the cessation of breathing caused by blockage of the air or the stopping of the breathing function. Typically, the individual will stop breathing for between about 15 seconds and about 30 seconds during an obstructive sleep apnea event. Mixed sleep apnea is another form of SDB that is a combination of OSA and CSA.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of SDB. CSR is a disorder of a patient’s respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

A wide variety of types of data can be used to monitor the health of individuals having any of the above types of sleep-related and/or respiratory disorders (or other disorders). However, it is often difficult to collect accurate data in a manner that does not interrupt or disturb the user’s sleep, or interfere with any treatment the user may be undergoing during sleep. Thus, it is advantageous to utilize a system for treatment that includes various sensors to generate and collect data, without disturbing the user, the user’s sleep, or the user’s treatment.

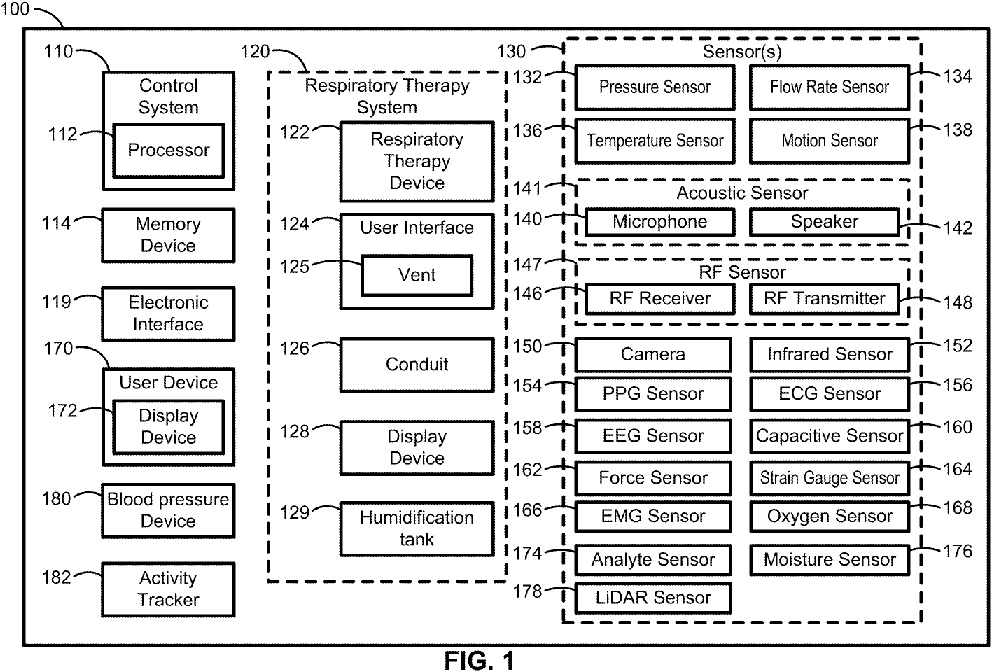

Referring to FIG. 1, a system 100, according to some implementations of the present disclosure, is illustrated. The system 100 is for providing a variety of different sensors related to a user’s use of a respiratory therapy system, among other uses. The system 100 includes a control system 110, a memory device 114, an electronic interface 119, one or more sensors 130, and one or more external devices 170. In some implementation, the system 100 further includes a respiratory therapy system 120 that includes a respiratory therapy device 122.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 (or any other control system) or a portion of the control system 110 such as the processor 112 (or any other processor(s) or portion(s) of any other control system), can be used to carry out one or more steps of any of the methods described and/or claimed herein. The control system 110 can be coupled to and/or positioned within, for example, a housing of the external device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of a respiratory therapy device 122 of the respiratory therapy system 120, within a housing of the external device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family medical history (such as a family history of insomnia or sleep apnea), an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the external device 170. In other implementations, the electronic interface 119 is coupled to or integrated (e.g., in a housing) with the control system 110 and/or the memory device 114.

As noted above, in some implementations, the system 100 optionally includes a respiratory therapy system 120 (also referred to as a respiratory pressure therapy system). The respiratory therapy system 120 can include a respiratory therapy device 122 (also referred to as a respiratory pressure therapy device), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user’s airways at a controlled target pressure that is nominally positive with respect to atmosphere throughout the user’s breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea), other respiratory disorders such as COPD, or other disorders leading to respiratory insufficiency, that may manifest either during sleep or wakefulness.

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about -20 L/min and about 150 L/min, while maintaining a positive pressure (relative to the ambient pressure). In some implementations, the control system 110, the memory device 114, the electronic interface 119, or any combination thereof can be coupled to and/or positioned within a housing of the respiratory therapy device 122.

The user interface 124 engages a portion of the user’s face and delivers pressurized air from the respiratory therapy device 122 to the user’s airway to aid in preventing the airway from narrowing and/or collapsing during sleep. This may also increase the user’s oxygen intake during sleep. Depending upon the therapy to be applied, the user interface 124 may form a seal, for example, with a region or portion of the user’s face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

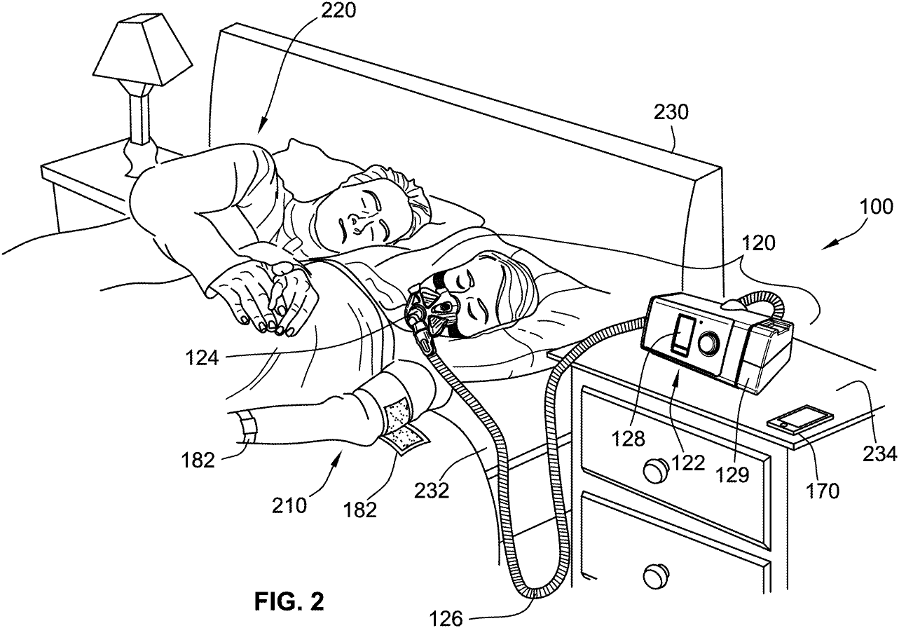

In some implementations, the user interface 124 is or includes a facial mask that covers the nose and mouth of the user (as shown, for example, in FIG. 2). Alternatively, the user interface 124 is or includes a nasal mask that provides air to the nose of the user or a nasal pillow mask that delivers air directly to the nostrils of the user. The user interface 124 can include a strap assembly that has a plurality of straps (e.g., including hook and loop fasteners) for positioning and/or stabilizing the user interface 124 on a portion of the user interface 124 on a desired location of the user (e.g., the face), and a conformal cushion (e.g., silicone, plastic, foam, etc.) that aids in providing an air-tight seal between the user interface 124 and the user. The user interface 124 can also include one or more vents 125 for permitting the escape of carbon dioxide and other gases exhaled by the user. In other implementations, the user interface 124 includes a mouthpiece (e.g., a night guard mouthpiece molded to conform to the user’s teeth, a mandibular repositioning device, etc.).

The conduit 126 allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation. Generally, the respiratory therapy system 120 forms an air pathway that extends between a motor of the respiratory therapy device 122 and the user and/or the user’s airway. Thus, the air pathway generally includes at least a motor of the respiratory therapy device 122, the user interface 124, and the conduit 126.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score or a therapy score (also referred to as a myAir™ score, such as described in WO 2016/061629, which is hereby incorporated by reference herein in its entirety), the current date/time, personal information for the user, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. In other implementations, the respiratory therapy device 122 or the conduit 126 can include a waterless humidifier. The waterless humidifier can incorporate sensors that interface with other sensor positioned elsewhere in the system 100.

The respiratory therapy system 120 can be used, for example, as a ventilator or a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based at least in part on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can include the display device 128, which can allow the user to interact with the respiratory therapy device 122. The respiratory therapy device 122 can also include the humidification tank 129, which stores the water used to humidify the pressurized air. The respiratory therapy device 122 can be positioned on a nightstand 234 that is directly adjacent to the bed 230 as shown in FIG. 2, or more generally, on any surface or structure that is generally adjacent to the bed 230 and/or the user 210. The user can also wear the blood pressure device 180 and the activity tracker 182 while lying on the mattress 232 in the bed 230.

Referring back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, an RF transmitter 148, a camera 150, an infrared (IR) sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a light detection and ranging (LiDAR) sensor 178, or any combination thereof. Generally, each of the one or sensors 130 are configured to output sensor data that is received and stored in the memory device 114 or one or more other memory devices. The sensors 130 can also include, an electrooculography (EOG) sensor, a peripheral oxygen saturation (SpO₂) sensor, a galvanic skin response (GSR) sensor, a carbon dioxide (CO₂) sensor, or any combination thereof.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the IR sensor 152, the PPG sensor 154, the ECG sensor 156, the EEG sensor 158, the capacitive sensor 160, the force sensor 162, the strain gauge sensor 164, the EMG sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example physiological data, acoustic data, or both, that is associated with a user of the respiratory therapy system 120 (such as the user 210 of FIG. 2), the respiratory therapy system 120, both the user and the respiratory therapy system 120, or other entities, objects, activities, etc. Physiological data generated by one or more of the sensors 130 can be used by the control system 110 to determine a sleep-wake signal associated with the user during the sleep session and one or more sleep-related parameters. The sleep-wake signal can be indicative of one or more sleep stages and/or sleep states, including sleep, wakefulness, relaxed wakefulness, micro-awakenings, or distinct sleep stages including a rapid eye movement (REM) stage (which can include both a typical REM stage and an atypical REM stage), a first non-REM stage (often referred to as “N1”), a second non-REM stage (often referred to as “N2”), a third non-REM stage (often referred to as “N3”), or any combination thereof. Methods for determining sleep stages and/or sleep states from physiological data generated by one or more of the sensors, such as sensors 130, are described in, for example, WO 2014/047310, US 2014/0088373, WO 2017/132726, WO 2019/122413, and WO 2019/122414, each of which is hereby incorporated by reference herein in its entirety.

The sleep-wake signal can also be timestamped to indicate a time that the user enters the bed, a time that the user exits the bed, a time that the user attempts to fall asleep, etc. The sleep-wake signal can be measured one or more of the sensors 130 during the sleep session at a predetermined sampling rate, such as, for example, one sample per second, one sample per 30 seconds, one sample per minute, etc. Examples of the one or more sleep-related parameters that can be determined for the user during the sleep session based at least in part on the sleep-wake signal include a total time in bed, a total sleep time, a total wake time, a sleep onset latency, a wake-after-sleep-onset parameter, a sleep efficiency, a fragmentation index, an amount of time to fall asleep, a consistency of breathing rate, a fall asleep time, a wake time, a rate of sleep disturbances, a number of movements, or any combination thereof.

Physiological data and/or acoustic data generated by the one or more sensors 130 can also be used to determine a respiration signal associated with the user during a sleep session. the respiration signal is generally indicative of respiration or breathing of the user during the sleep session. The respiration signal can be indicative of, for example, a respiration rate, a respiration rate variability, an inspiration amplitude, an expiration amplitude, an inspiration-expiration amplitude ratio, an inspiration-expiration duration ratio, a number of events per hour, a pattern of events, pressure settings of the respiratory therapy device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, an elevated stress level, etc.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of the user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user, a skin temperature of the user, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. The motion sensor 138 can be used to detect motion or acceleration associated with arterial pulses, such as pulses in or around the face of the user and proximal to the user interface 124, and configured to detect features of the pulse shape, speed, amplitude, or volume.

The microphone 140 outputs acoustic data that can be stored in the memory device 114 and/or analyzed by the processor 112 of the control system 110. The acoustic data generated by the microphone 140 is reproducible as one or more sound(s) during a sleep session (e.g., sounds from the user) to determine (e.g., using the control system 110) one or more sleep-related parameters, as described in further detail herein. The acoustic data from the microphone 140 can also be used to identify (e.g., using the control system 110) an event experienced by the user during the sleep session, as described in further detail herein. In other implementations, the acoustic data from the microphone 140 is representative of noise associated with the respiratory therapy system 120. The microphone 140 can be coupled to or integrated in the respiratory therapy system 120 (or the system 100) generally in any configuration. For example, the microphone 140 can be disposed inside the respiratory therapy device 122, the user interface 124, the conduit 126, or other components. The microphone 140 can also be positioned adjacent to or coupled to the outside of the respiratory therapy device 122, the outside of the user interface 124, the outside of the conduit 126, or outside of any other components. The microphone 140 could also be a component of the external device 170 (e.g., the microphone 140 is a microphone of a smart phone). The microphone 140 can be integrated into the user interface 124, the conduit 126, the respiratory therapy device 122, or any combination thereof. In general, the microphone 140 can be located at any point within or adjacent to the air pathway of the respiratory therapy system 120, which includes at least the motor of the respiratory therapy device 122, the user interface 124, and the conduit 126. Thus, the air pathway can also be referred to as the acoustic pathway.

The speaker 142 outputs sound waves that are audible to the user. The speaker 142 can be used, for example, as an alarm clock or to play an alert or message to the user (e.g., in response to an event). In some implementations, the speaker 142 can be used to communicate the acoustic data generated by the microphone 140 to the user. The speaker 142 can be coupled to or integrated in the respiratory therapy device 122, the user interface 124, the conduit 126, or the external device 170.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and/or frequency, and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user or a bed partner of the user (such as bed partner 220 in FIG. 2). Based at least in part on the data from the microphone 140 and/or the speaker 142, the control system 110 can determine a location of the user and/or one or more of the sleep-related parameters described in herein, such as, for example, a respiration signal, a respiration rate, an inspiration amplitude, an expiration amplitude, an inspiration-expiration ratio, a number of events per hour, a pattern of events, a sleep stage, pressure settings of the respiratory therapy device 122, or any combination thereof. In this context, a SONAR sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the external device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a RADAR sensor). In some such implementations, the RF sensor 147 includes a control circuit. The specific format of the RF communication could be WiFi, Bluetooth, etc.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based at least in part on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user enters the user’s bed (such as bed 230 in FIG. 2), and to determine a time when the user exits the bed 230. The camera 150 can also be used to track eye movements, pupil dilation (if one or both of the user’s eyes are open), blink rate, or any changes during REM sleep. The camera 150 can also be used to track the position of the user, which can impact the duration and/or severity of apneic episodes in users with positional obstructive sleep apnea.

The IR sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user and/or movement of the user. The IR sensor 152 can also be used in conjunction with the camera 150 when measuring the presence, location, and/or movement of the user. The IR sensor 152 can detect infrared light having a wavelength between about 700 nm and about 1 mm, for example, while the camera 150 can detect visible light having a wavelength between about 380 nm and about 740 nm.

The IR sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user and/or movement of the user. The IR sensor 152 can also be used in conjunction with the camera 150 when measuring the presence, location, and/or movement of the user. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user, embedded in clothing and/or fabric that is worn by the user, embedded in and/or coupled to the user interface 124 and/or its associated headgear (e.g., straps, etc.), etc.

The ECG sensor 156 outputs physiological data associated with electrical activity of the heart of the user. In some implementations, the ECG sensor 156 includes one or more electrodes that are positioned on or around a portion of the user during the sleep session. The physiological data from the ECG sensor 156 can be used, for example, to determine one or more of the sleep-related parameters described herein.

The EEG sensor 158 outputs physiological data associated with electrical activity of the brain of the user. In some implementations, the EEG sensor 158 includes one or more electrodes that are positioned on or around the scalp of the user during the sleep session. The physiological data from the EEG sensor 158 can be used, for example, to determine a sleep stage and/or a sleep state of the user at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user. The data output by the analyte sensor 174 can be stored in the memory device 114 and used by the control system 110 to determine the identity and concentration of any analytes in the user’s breath. In some implementations, the analyte sensor 174 is positioned near a mouth of the user to detect analytes in breath exhaled from the user’s mouth. For example, when the user interface 124 is a facial mask that covers the nose and mouth of the user, the analyte sensor 174 can be positioned within the facial mask to monitor the user mouth breathing. In other implementations, such as when the user interface 124 is a nasal mask or a nasal pillow mask, the analyte sensor 174 can be positioned near the nose of the user to detect analytes in breath exhaled through the user’s nose. In still other implementations, the analyte sensor 174 can be positioned near the user’s mouth when the user interface 124 is a nasal mask or a nasal pillow mask. In this implementation, the analyte sensor 174 can be used to detect whether any air is inadvertently leaking from the user’s mouth. In some implementations, the analyte sensor 174 is a volatile organic compound (VOC) sensor that can be used to detect carbon-based chemicals or compounds, such as carbon dioxide. In some implementations, the analyte sensor 174 can also be used to detect whether the user is breathing through their nose or mouth. For example, if the data output by an analyte sensor 174 positioned near the mouth of the user or within the facial mask (in implementations where the user interface 124 is a facial mask) detects the presence of an analyte, the control system 110 can use this data as an indication that the user is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user’s face, near the connection between the conduit 126 and the user interface 124, near the connection between the conduit 126 and the respiratory therapy device 122, etc.). Thus, in some implementations, the moisture sensor 176 can be coupled to or integrated into the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user, for example the air inside the user’s bedroom. The moisture sensor 176 can also be used to track the user’s biometric response to environmental changes.

One or more LiDAR sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the external device 170, or any combination thereof. For example, the acoustic sensor 141 and/or the RF sensor 147 can be integrated in and/or coupled to the external device 170. In such implementations, the external device 170 can be considered a secondary device that generates additional or secondary data for use by the system 100 (e.g., the control system 110) according to some aspects of the present disclosure. In some implementations, the pressure sensor 132 and/or the flow rate sensor 134 are integrated into and/or coupled to the respiratory therapy device 122. In some implementations, at least one of the one or more sensors 130 is not coupled to the respiratory therapy device 122, the control system 110, or the external device 170, and is positioned generally adjacent to the user during the sleep session (e.g., positioned on or in contact with a portion of the user, worn by the user, coupled to or positioned on the nightstand, coupled to the mattress, coupled to the ceiling, etc.). More generally, the one or more sensors 130 can be positioned at any suitable location relative to the user such that the one or more sensors 130 can generate physiological data associated with the user and/or the bed partner 220 during one or more sleep session.

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, an average duration of events, a range of event durations, a ratio between the number of different events, a sleep stage, an apnea-hypopnea index (AHI), or any combination thereof. The one or more events can include snoring, apneas, central apneas, obstructive apneas, mixed apneas, hypopneas, an intentional user interface leak, an unintentional user interface leak, a mouth leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The external device 170 includes a display device 172. The external device 170 can be, for example, a mobile device such as a smart phone, a tablet, a laptop, or the like. Alternatively, the external device 170 can be an external sensing system, a television (e.g., a smart television) or another smart home device (e.g., a smart speaker(s) such as Google Home, Amazon Echo, Alexa etc.). In some implementations, the external device 170 is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the external device 170. In some implementations, one or more external devices 170 can be used by and/or included in the system 100.

The blood pressure device 180 is generally used to aid in generating physiological data for determining one or more blood pressure measurements associated with a user. The blood pressure device 180 can include at least one of the one or more sensors 130 to measure, for example, a systolic blood pressure component and/or a diastolic blood pressure component.

In some implementations, the blood pressure device 180 is a sphygmomanometer including an inflatable cuff that can be worn by a user and a pressure sensor (e.g., the pressure sensor 132 described herein). For example, as shown in the example of FIG. 2, the blood pressure device 180 can be worn on an upper arm of the user. In such implementations where the blood pressure device 180 is a sphygmomanometer, the blood pressure device 180 also includes a pump (e.g., a manually operated bulb) for inflating the cuff. In some implementations, the blood pressure device 180 is coupled to the respiratory therapy device 122 of the respiratory therapy system 120, which in turn delivers pressurized air to inflate the cuff. More generally, the blood pressure device 180 can be communicatively coupled with, and/or physically integrated in (e.g., within a housing), the control system 110, the memory device 114, the respiratory therapy system 120, the external device 170, and/or the activity tracker 182.

The activity tracker 182 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 182 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 182 is a wearable device that can be worn by the user, such as a smartwatch, a wristband, a ring, or a patch. For example, referring to FIG. 2, the activity tracker 182 is worn on a wrist of the user. The activity tracker 182 can also be coupled to or integrated a garment or clothing that is worn by the user. Alternatively, still, the activity tracker 182 can also be coupled to or integrated in (e.g., within the same housing) the external device 170. More generally, the activity tracker 182 can be communicatively coupled with, or physically integrated in (e.g., within a housing), the control system 110, the memory device 114, the respiratory therapy system 120, the external device 170, and/or the blood pressure device 180.

While the control system 110 and the memory device 114 are described and shown in FIG. 1 as being a separate and distinct component of the system 100, in some implementations, the control system 110 and/or the memory device 114 are integrated in the external device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for analyzing data associated with a user’s use of the respiratory therapy system 120, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the external device 170. As yet another example, a third alternative system includes the control system 110, the memory device 114, the respiratory therapy system 120, at least one of the one or more sensors 130, and the external device 170. As a further example, a fourth alternative system includes the control system 110, the memory device 114, the respiratory therapy system 120, at least one of the one or more sensors 130, the external device 170, and the blood pressure device 180 and/or activity tracker 182. Thus, various systems for analyzing data associated with a user’s use of the respiratory therapy system 120 can be formed using any portion or portions of the components shown and described herein and/or in combination with one or more other components.

As used herein, a sleep session can be defined in a number of ways based at least in part on, for example, an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the external device 170 (FIG. 1) to manually initiate or terminate the sleep session.

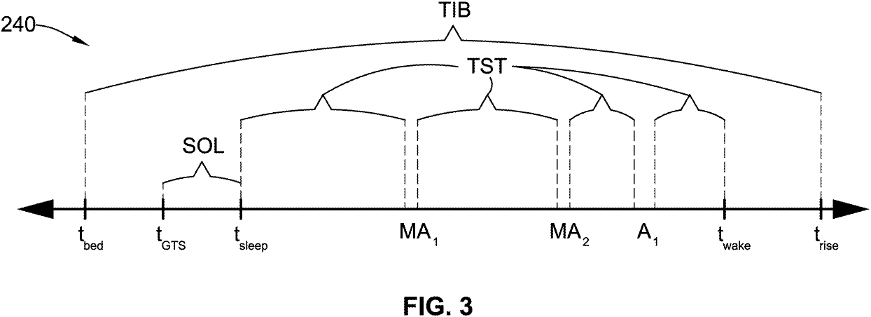

Referring to FIG. 3, an exemplary timeline 240 for a sleep session is illustrated. The timeline 240 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the external device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user’s sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 240 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

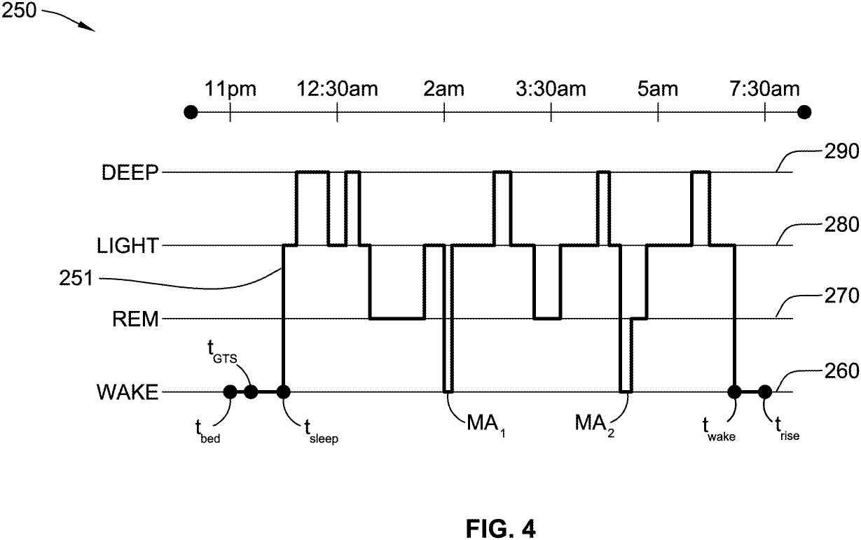

Referring to FIG. 4, an exemplary hypnogram 250 corresponding to the timeline 240 (FIG. 3), according to some implementations, is illustrated. As shown, the hypnogram 250 includes a sleep-wake signal 251, a wakefulness stage axis 260, a REM stage axis 270, a light sleep stage axis 280, and a deep sleep stage axis 290. The intersection between the sleep-wake signal 251 and one of the axes 260-290 is indicative of the sleep stage at any given time during the sleep session.

The sleep-wake signal 251 can be generated based at least in part on physiological data associated with the user (e.g., generated by one or more of the sensors 130 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 250 is shown in FIG. 4 as including the light sleep stage axis 280 and the deep sleep stage axis 290, in some implementations, the hypnogram 250 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 114.

The hypnogram 250 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 138, the microphone 140, the camera 150, or any combination thereof. The go-to-sleep time can be determined based at least in part on, for example, data from the motion sensor 138 (e.g., data indicative of no movement by the user), data from the camera 150 (e.g., data indicative of no movement by the user and/or that the user has turned off the lights), data from the microphone 140 (e.g., data indicative of the using turning off a TV), data from the external device 170 (e.g., data indicative of the user no longer using the external device 170), data from the pressure sensor 132 and/or the flow rate sensor 134 (e.g., data indicative of the user turning on the respiratory therapy device 122, data indicative of the user donning the user interface 124, etc.), or any combination thereof.

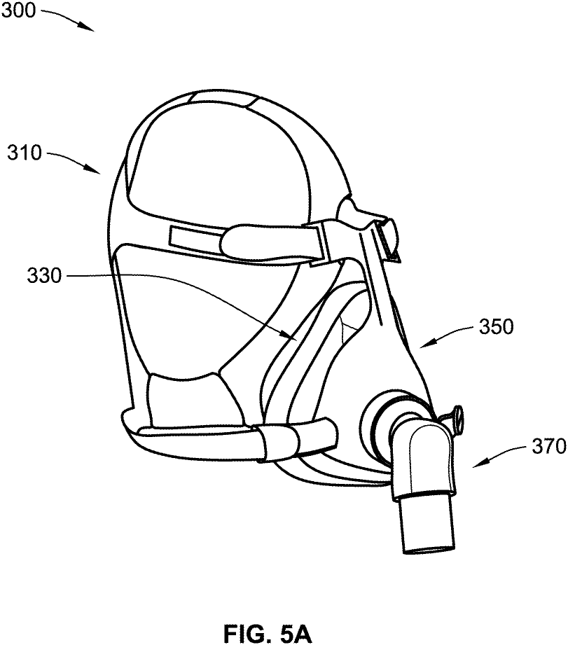

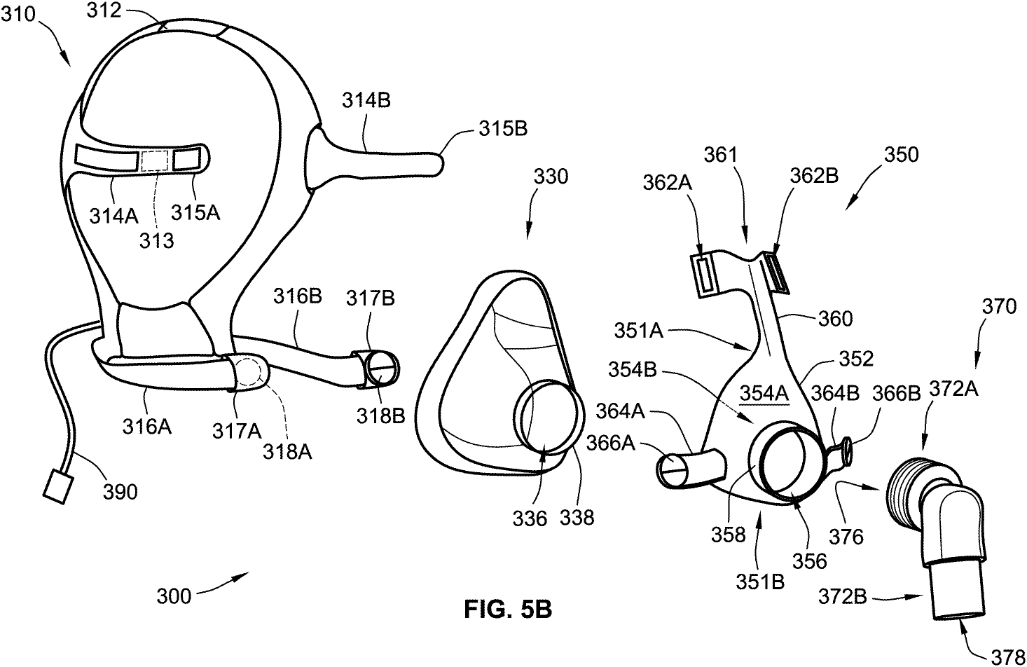

A user interface 300 is illustrated in FIGS. 5A and 5B. User interface 300 may be the same as or similar to user interface 124 as discussed herein with respect to FIGS. 1 and 2, and can be used in conjunction with any of the above-described components or features of system 100, including respiratory therapy system 120 and respiratory therapy device 122. The user interface 300 includes a strap assembly 310, a cushion 330, a frame 350, and a connector 370. The strap assembly 310 is configured to be positioned generally about at least a portion of the user’s head when the user wears the user interface 300. The strap assembly 310 can be coupled to the frame 350 and positioned on the user’s head such that the user’s head is positioned between the strap assembly 310 and the frame 350.

In some implementations, the cushion 330 is positioned between the user’s face and the frame 350 to form a seal on the user’s face. A first end portion 372A of the connector 370 is coupled to the frame 350, while a second end portion 372B of the connector 370 can be coupled to a conduit (such as conduit 126). In turn, the conduit can be coupled to the air outlet of a respiratory therapy device (such as respiratory therapy device 122). A blower motor in the respiratory therapy device is operable to generate a flow of pressurized air out of the air outlet, to thereby provide pressurized air to the user. The pressurized air can flow from the respiratory therapy device and through the conduit, the connector 370, the frame 350, and the cushion 330, until the air reaches the user’s airway through the user’s mouth, nose, or both.

The strap assembly 310 is formed from a rear portion 312, a pair of upper straps 314A and 314B, and a pair of lower straps 316A and 316B. The rear portion 312 of the strap assembly is generally positioned behind the user’s head when the user wears the user interface 300. The upper straps 314A, 314B and the lower straps 316A, 316B extend from the rear portion 312 toward the front of the user’s face. In the illustrated implementation, the rear portion 312 has a circular shape. However, the rear portion 312 may also have other shapes. The rear portion 312, the upper straps 314A, 314B, and the lower straps 316A, 316B can be formed or woven from a generally stretchy or resilient material, such as fabric, elastic, rubber, etc., or any combination of materials. In some implementations, the electrical wires or traces may extend through the interior of a portion of the strap assembly 310. This portion of the strap assembly 310 may generally form around the electrical wires or traces, or may have a hollow interior or channel through which electrical wires or traces extend, as discussed in further detail below.

The upper straps 314A, 314B and the lower straps 316A, 316B each have first ends originating at the rear portion 312, and second ends that couple to the frame 350. When the user wears the user interface 300, the tension provided by the strap assembly 310 holds the frame 350 to the user’s face, thus securing the user interface 300 to the user’s head.

In some implementations, a tension sensor can be embedded in one of the straps of the strap assembly. For example, FIG. 5B illustrates a tension sensor 313 embedded in upper strap 314A. The tension sensor 313 is configured to measure tension in the straps of the user interface 124. As discussed, the user interface 124 is generally fasted to the user 210’s head using straps that can be tightened using Velcro™ or some other fastener. The tension sensor 313 can sense the tension in the straps, which can then be used to inform and/or instruct the user 210 about the correct fitting of the user interface 124. The tension sensor 313 can be integrated into yarn, fiber, wire, carbon fiber, warps, webs. etc. As the tension in the strap increases or decreases, the sensor element of the tension sensor 313 is deflected, causing a change in the voltage of an output signal. The tension sensor 313 can have high elasticity and low resistance, and the ability to be washed. In some implementations, the tension sensor 313 measures the diameter of an inflatable body by the principles of respiratory inductance plethysmography. The tensor sensor 313 can also be an electric impedance plethysmography sensor, a magnetometer, a strain gauge sensor, or be made of piezo-resistive material displacement sensor.

The frame 350 is generally formed from a body 352 that defines a first surface 354A and an opposing second surface 354B. When the user wears the user interface 300, the first surface 354A faces away from the user’s face, while the second surface 354B faces toward the user’s face. The frame also defines an annular aperture 356 into which the cushion 330 and the connector 370 can be inserted, to thereby physically couple the cushion 330 and the connector 370 to the frame 350.

The cushion 330 can be coupled to the inside of the frame 350 adjacent to the second surface 354B, such that the cushion 330 is positioned between the user’s face and the frame 350. The cushion 330 can be made from the same as or similar to the cushion of user interface 124, and thus can be formed of a conformal material that forms an air-tight seal with the user’s face. The cushion 330 defines an aperture 336, and includes an annular projection 338 extending from the cushion 330 about the aperture 336 of the cushion. The annular projection 338 is inserted into the annular aperture 356 of the frame 350, such that the annular aperture 336 of the cushion 330 overlaps with the annular aperture 356 of the frame 350. In some implementations, the annular projection 338 of the cushion 330 is releasably secured to the body 352 of the frame 350 via a friction fit between the annular projection 338 and the body 352 around the annular aperture 356.

In other implementations, the annular projection 338 and the frame 350 can have mating features that mate with each other to secure the cushion 330 to the frame 350. For example, the annular projection 338 of the cushion 330 may include an outwardly-extending peripheral flange, and the body 352 of the frame 350 can include a corresponding inwardly-extending peripheral flange about the annular aperture 356. When the annular projection 338 of the cushion 330 is inserted into the annular aperture 356 of the frame 350, the peripheral flanges can slide or snap past each other, to thereby secure the cushion 330 to the frame 350. In additional implementations, the cushion 330 is held in place by the tension provided by the strap assembly 310, and is not physically coupled to the frame 350. In still other implementations, the cushion 330 and the frame 350 can be formed as a single integral piece.

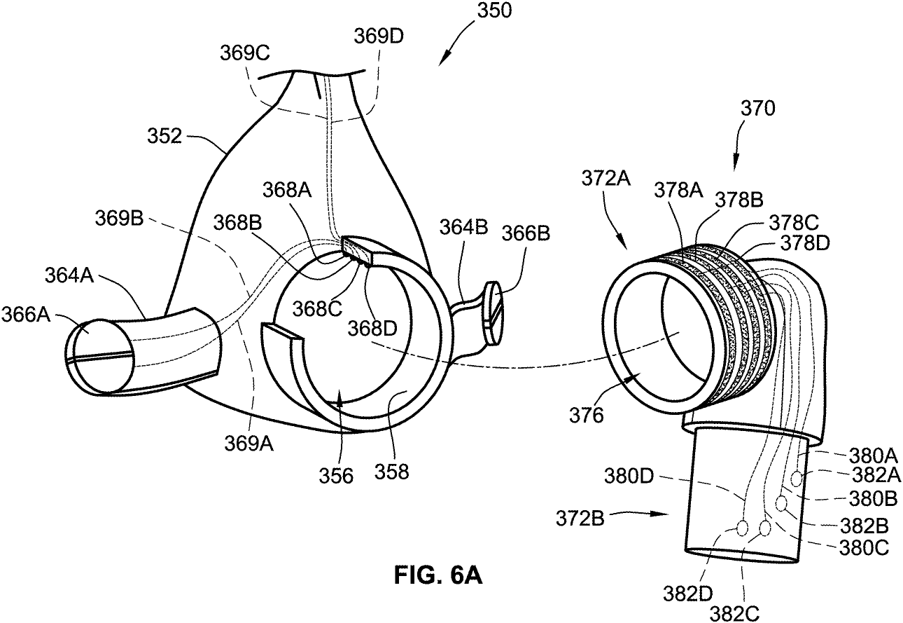

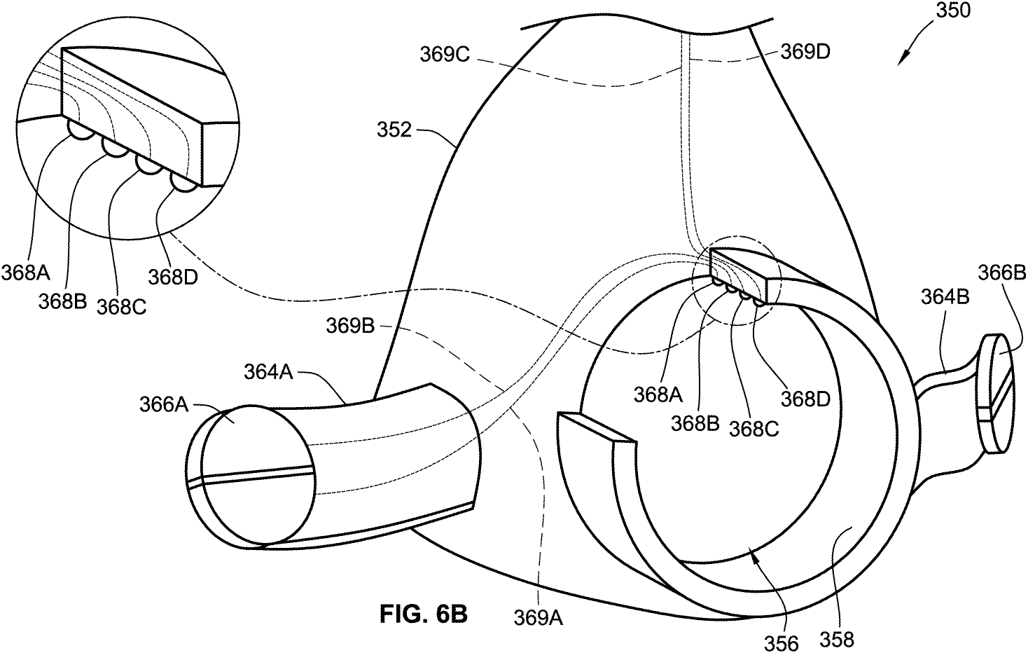

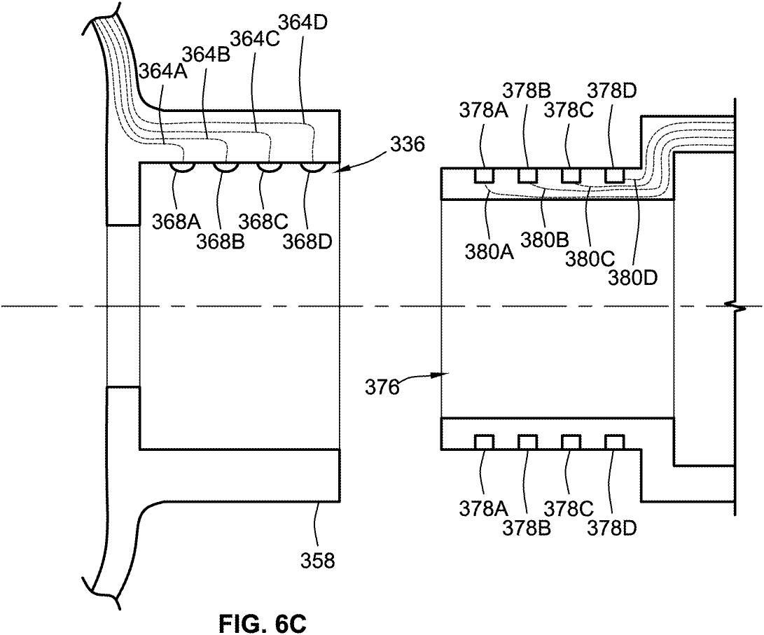

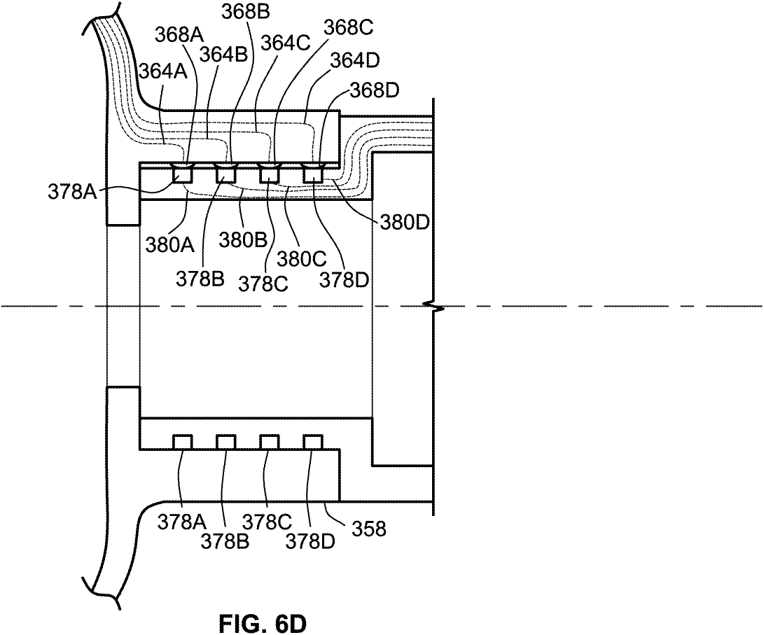

The connector 370 can be coupled to the opposite side of the frame 350 in a similar manner to the cushion 330. The first end portion 372A of the connector 370 has a generally cylindrical shape and can be inserted into the annular aperture 356 of the frame 350, such that a hollow interior 376 of the end portion 372A (see FIG. 6A) overlaps with the annular aperture 356, and the aperture 336 of the cushion 330. The opposing second end portion 372B of the connector 370 is then coupled to the conduit, such that the user’s face (including the user’s mouth and/or nose) is in fluid communication with the conduit through the cushion 330, the frame 350, and the connector 370.

The first end portion 372A of the connector 370 is generally annular-shaped, and fits into the annular aperture 356 of the frame 350. The frame 350 also includes an annular projection 358 that extends from the second surface 354B of the frame 350 and is formed about the annular aperture 356. When the first end portion 372A is inserted into the annular aperture 356 of the frame 350, an inner surface of the annular projection 358 overlaps with an outer surface of the first end portion 372AA of the connector 370.

In some implementations, a friction fit between the annular projection 358 and the first end portion 372A secures the connector 370 to the frame 350. In other implementations, the connector 370 can include a fastener configured to secure the connector 370 to the frame 350. In one example, the annular projection 358 has an outwardly-extending peripheral flange, and the fastener is one or more deflectable latches formed on the first end portion 372A of the connector 370. As the first end portion 372A slides is inserted within the annular projection 358, the deflectable latch slides over the peripheral flange such that the deflectable latch is positioned outside of the annular projection 358. As the deflectable latch passes by the peripheral flange, the peripheral flange pushes the deflectable latch away from the annular projection 358. The deflectable latch then returns to its original position, such that the connector 370 cannot be removed from the frame 350 without manually deflecting the deflectable latch away from the annular projection 358.

The frame 350 includes a T-shaped extension strip 360 extending upward from an upper end 351A of the body 352. In some implementations, the extension strip 360 is integrally formed with the body 352. In other implementations, the extension strip 360 is a separate component that is coupled to the body 352. When the user wears the user interface 300, the extension strip 360 generally extends up to the user’s forehead. In some implementations, the extension strip 360 includes a cooling portion or mechanism that contacts and cools the user 210’s forehead, which can help users with insomnia fall asleep.

The lower straps 316A, 316B extend toward the frame 350 from the rear portion 312 of the strap assembly 310, and are coupled to opposite sides of a lower end 351B of the body 352. The upper straps 314A, 314B extend toward the frame 350 from the rear portion 312 of the strap assembly 310, and are coupled to opposite sides of the upper end 361 extension strip 360 (e.g., the generally horizontal “cross” of the T). The frame 350 can include a variety of different strap attachment points to couple with the upper straps 314A, 314B and the lower straps 316A, 316B.

One type of strap attachment point is shown in the extension strip 360. The upper end 361 of the extension strip 360 includes two apertures 362A, 362B. These apertures can be integrally formed in the extension strip 360 itself, or may be formed as part of a separate component or piece that is coupled to the extension strip 360. The apertures 362A, 362B are shaped to allow the ends 315A, 315B of the upper straps 314A, 314B to be inserted through the apertures 362A, 362B. The ends 315A, 315B can then loop back and fasten to remainder of the upper straps 314A, 314B via any suitable mechanism, such as Velcro™, adhesive, etc. The upper straps 314A, 314B are thus secured to the extension strip 360 of the frame 350.

The frame 350 is shown with a different type of strap attachment point used to couple the lower straps 316A, 316B to the frame 350. The frame 350 includes two lateral strips 364A, 364B extending away from opposite ends of the lower end 351B of the body 352. The first end of each lateral strip 364A, 364B is coupled to the body 352, and a corresponding magnet 366A, 366B is disposed at the second end of each lateral strip 364A, 364B. A magnet 318A is coupled to end 317A of lower strap 316A, while a magnet 318B is coupled to end 317B of lower strap 316B. Magnet 318A can be secured to magnet 366A via magnetic attraction, while magnet 318B can be secured to magnet 366B via magnetic attraction, to thereby couple the lower straps 316A, 316B to the body 352 of the frame 350.

In some implementations, the frame 350 does not include the extension strip 360, and the upper straps 314A, 314B are instead coupled to the frame, above the lateral strips 364A, 364B. The upper straps 314A, 314B in these implementations extend past the user 210’s temples and around to the rear of the user 210’s head. The frame 350 may include upper lateral strips which the upper straps 314A, 314B are coupled to.

The user interface 300 can also include one or more sensors 390. While FIG. 5B generally only shows a single sensor, any number of sensors can be coupled to the strap assembly 310. In some implementation, the one or more sensors 390 are coupled to the strap assembly 310, and are configured to abut a target area of the user when the user wears the user interface 300. The target area could be the user’s forehead, temple, throat, neck, ear, etc. Generally, the one or more sensors 390 abutting the target area can include sensors that directly contact the target area of the user (e.g., the sensors touch the target area of the user), and/or sensors that do not directly contact the user (e.g., the sensors are separated from the target area of the user in some fashion).

In some implementations, the one or more sensors 390 are contact sensors, which can include an electroencephalography (EEG) sensor, an electrocardiogram (ECG) sensor, an electromyography (EMG) sensor, an electrooculography (EOG) sensor, an acoustic sensor, a peripheral oxygen saturation (SpO₂) sensor, a galvanic skin response (GSR) sensor, or any combination thereof. The contact sensors can directly contact the target area of the user, or may contact a layer of material positioned between the contact sensors and the target area, such as fabric (which could be the strap assembly 310), silicone (which could be the cushion 330), foam (which could be the cushion 330), plastic (which could be the frame 350), etc. In some implementations, the one or more sensors 390 are non-contact sensors, which can include a carbon dioxide (CO₂) sensor (to measure CO₂ concentration), an oxygen (O₂) sensor (to measure O₂ concentration), a pressure sensor, a temperature sensor, a motion sensor, a microphone, an acoustic sensor, a flow sensor, a tension sensor, or any combination thereof. Generally, these non-contact sensors can be spaced apart from the target area, such that there is air (or any other material) positioned between the one or more sensors 390 and the target area.

In other implementations, the one or more sensors 390 are not coupled to the strap assembly 310, but are instead located at other positions within the user interface 300, such as within the connector 370. The one or more sensors 390 can be any one or more of the sensors 130 described herein with respect to FIG. 1, and can additionally or alternatively include other types of sensors as well. In some implementations, the one or more sensors 390 can include one or more non-contact sensors and one or more contact sensors. In some of these implementations, the non-contact sensor is not coupled to the strap assembly 310, but is instead disposed in the cushion 330, the frame 350, or the connector 370. Moreover, the user interface 300 can include multiple non-contact sensors disposed in any combination of these locations. In one example, one of the one or more sensors 390 is coupled to the frame 350, and contacts the target area via the cushion 330. In this example, the sensor could be positioned at or near the surface of the cushion 330. Thus, the one or more sensors 390 can include any combination of sensors that (i) directly touch the target area or (ii) are spaced apart from the target area and are separated from the target area by air or some other material. The one or more sensors 390 can include any combination of contact sensors and non-contact sensors.

Generally, the one or more sensors 390 of the user interface 300 need to be electrically connected to a control system and a memory device (such as control system 110 and memory device 114 of system 100) in order to transmit data to the control system and memory device. These data can be used to modify the operation of the respiratory therapy device, and can also be used for other purposes. In order send the data from the one or more sensors 390 to the control system and memory device, the one or more sensors 390 can be electrically connected to various parts of the user interface 300, including the frame 350 and the connector 370. Data from the one or more sensors 390 can be transmitted using the electrical connection between the one or more sensors 390, the frame 350, and the connector 370. Thus, wherever the one or more sensors 390 are located in the user interface 300, the one or more sensors 390 need to be able to be electrically connected to the control system and the memory device.

FIGS. 6A and 6B show the electrical connection between the frame 350 and the connector 370. The frame 350 includes electrical contacts 368A, 368B, 368C, and 368D disposed on the inside of the annular projection 358. The electrical contacts 368A-368D can be formed on the inner surface of the annular projection 358, or may extend radially inward from the inner surface of the annular projection 358. In FIGS. 6A and 6B, a portion of the annular projection 358 has been removed to better show the electrical contacts 368A-368D. The connector 370 includes corresponding electrical contacts 378A, 378B, 378C, 378D disposed on the surface of the annular-shaped end portion 372A.

When the end portion 372A of the connector 370 is inserted into the annular aperture 356 of the frame 350, each electrical contact of the frame 350 physically contacts one of the electrical contacts of the connector 370, such that the frame 350 is electrically connected to the connector 370. Thus, electrical contact 368A is physically and electrically connected to electrical contact 378A, electrical contact 368B is physically and electrically connected to electrical contact 378B, electrical contact 368C is physically and electrically connected to electrical contact 378C, and electrical contact 368D is physically and electrically connected to electrical contact 378D. Thus, the connector 370 can be physically and electrically connected to the frame 350.

In the illustrated implementation, each electrical contact 378A-378D of the connector 370 is an annular electrical contact that forms a ring on the surface of the end portion 372A of the connector 370. Annular electrical contacts 378A-378D may be formed on the surface of end portion 372A, or may extend radially outward from the surface of end portion 372A. The electrical contacts 368A-368D of the frame 350 are formed as single electrical pads, each located at one location on the inner surface of the annular projection 358. The electrical contacts 368A-368D may be formed on the inner surface of the annular projection 358, or may be formed as pins that extend radially inward from the inner surface of the annular projection 358. The annular shapes of electrical connectors 378A-378D ensures that if the connector 370 is rotated relative to the frame 350 once the end portion 372A is inserted into the annular aperture 356 of the frame 350, some portion of each electrical contact 378A-378D will always be physically touching its corresponding electrical contact 368A-368D of the frame 350.

In other implementations however, the electrical contacts 368A-368D of the frame 350 may have annular shapes that form rings on the inner surface of the annular projection 358, while the electrical contacts 378A-378D of the connector 370 are single electrical pads, each located at one location on the outer surface of end portion 372A. In still other implementations, electrical contacts 368A-368D and electrical contacts 378A-378D are all annular electrical contacts. In further implementations, electrical contacts 368A-368D and electrical contacts 378A-378D are all formed as single electrical pads. In some implementations, electrical contacts 368A-368D and electrical contacts 378A-378D are at least partially annular, meaning that they can form partial rings. The rings can be quarter-rings (e.g., 90°), half-rings (e.g., 180°), three-quarter rings (e.g., 270°), or any other partially annular arrangement.

The connector 370 includes electrical contacts 382A-382D located at the other end portion 372B. Electrical contact 382A is electrically connected to electrical contact 378A via an electrical pathway 380A formed in the hollow interior 376 of the connector. Electrical contact 382B is electrically connected to electrical contact 378B via an electrical pathway 380B formed in the hollow interior 376 of the connector. Electrical contact 382C is electrically connected to electrical contact 378C via an electrical pathway 380C formed in the hollow interior 376 of the connector. Electrical contact 382D is electrically connected to electrical contact 378D via an electrical pathway 380D formed in the hollow interior 376 of the connector.

The electrical pathways 380A-380D can be formed in a variety of manners. In some implementations, electrical pathways 380A-380D are electrical traces formed on the inner surface of the hollow interior 376 of the connector 370, or within the connector 370 itself. In other implementations, electrical pathways 380A-380D are formed from wires positioned inside the hollow interior 376 of the connector 370. The second end portion 372A of the connector 370 can be inserted into the conduit, which can have similar electrical contacts. In turn, the electrical contacts of the conduit may be electrically connected to the control system and memory device when the conduit is coupled to the respiratory therapy device. Thus, the connector 370 can be physically and electrically coupled to a conduit.

The electrical contacts 368A-368D of the annular projection 358 can be electrically connected to the strap attachment points of the frame 350. Electrical pathways 369A and 369B extend from electrical contacts 368A and 368B, respectively, through lateral strip 364A, and out to magnet 366A. As discussed further herein, lateral strip 364A and magnet 366A can be electrically connected to one of the straps of the strap assembly 310. In a similar manner, electrical pathways 369C and 369D extend from electrical contacts 368C and 368D, respectively, upwards through the extension strip 360. While not shown in FIGS. 6A and 6B, one or more electrical pathways may also extend through lateral strip 364B out to magnet 366B.

Electrical pathways 369A-369D can be formed in a variety of different manners. In some implementations, electrical pathways 369A-369D are formed by wires positioned between adj acent to the second surface of the body 352, between the frame 350 and the cushion 330. In other implementations, electrical pathways 369A-369D can be formed by electrical traces that are formed on the second surface of the body 352, or formed within the body 352 between the first surface and the second surface.

Electrical pathways 3689-369D shown in FIGS. 6A and 6B are example implementations. In other implementations, any number of electrical pathways can be formed between the electrical contacts 368A-368D of the annular projection 358 and any point on the frame 350. For example, some of the electrical contacts 368A-368D can be electrically connected to lateral strip 364B and magnet 366B, instead of or in addition to electrical connections to lateral strip 364A and magnet 366A, and extension strip 360.

FIG. 6C shows a cross-sectional view of the annular projection 358 of the frame 350 and the first end portion 372A of the connector 370, prior to the first end portion 372A being inserted into the annular aperture 356 of the frame 350. FIG. 6D shows a cross-section view after the first end portion 372A is inserted into the annular aperture 356 of the frame 350. Electrical contacts 368A-368D of the annular projection 358 are formed as single pads on the inner surface of the annular projection 358. Electrical contacts 378A-378D of the connector 370 are annular electrical contacts formed as rings on the outer surface of the first end portion 372. Electrical pathways 369A-369D of the frame 350 are electrically connected to electrical contacts 368A-368D, respective. Electrical pathways 380A-380D of the connector 370 are electrically connected to electrical contacts 378A-378D, respective.

Once the first end portion 372A is inserted into the annular aperture 356 of the frame 350, annular electrical contacts 378A-378D come into contact with electrical contacts 368A-368D, thereby electrically connecting the two sets of electrical contacts. In turn, electrical pathways 369A-369D are electrically connected to electrical pathways 378A-378D. Because electrical contacts 378A-378D are annular-shaped, the connector 370 can be rotated through any number of revolutions, and the connector 370 will remain electrically connected to the frame 350.

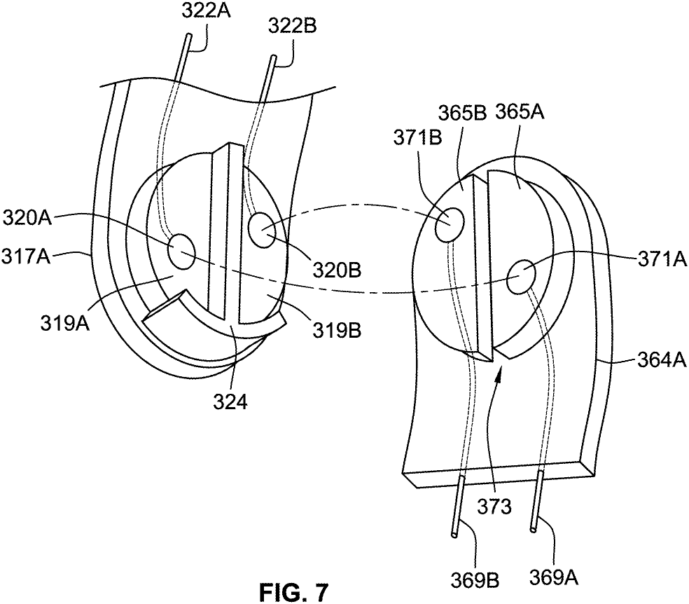

FIGS. 7A and 7B illustrate an implementation for electrically connecting the strap attachment points of the frame 350 to straps of the strap assembly 310. FIG. 7A shows only the strap attachment point formed by lateral strip 364A. However, this implementation can be used for lateral strip 364B, or for other strap attachment points of the frame 350.

As shown in FIG. 7A, electrical pathways 369A and 369B extend through lateral strip 364A and terminate at magnets 365A and 365B. Magnets 365A and 365B are generally the same as or similar to magnet 366A in FIGS. 6A and 6B, except that the magnet is formed from two smaller magnets 365A and 365B. Electrical pathway 369A terminates at an electrical contact 371A that is adjacent to magnet 365A. Similarly, electrical pathway 369B terminates at an electrical contact 371B that is adjacent to magnet 365B. In the illustrated implementation, electrical contact 371A is generally flush with the surface of magnet 365A, while electrical contact 371B is generally flush with the surface of magnet 365B.

The end 317A of lower strap 316A is generally formed in the same fashion. Magnets 319A and 319B are mounted at the end 317A of the lower strap 316A. Magnets 319A and 319B are generally the same as magnet 318A shown in FIG. 5B, except that the magnet is formed from two smaller magnets 319A and 319B. Magnet 319A includes electrical contact 320A that is generally flush with the surface of magnet 319A. Similarly, magnet 319B includes electrical contact 320B that is generally flush with the surface of magnet 319B. Electrical contact 320A is electrically connected to electrical pathway 322A, while electrical contact 320B is electrically connected to electrical pathway 322B. Electrical pathways 322A, 322B extend through the lower strap 316A, to any desired point along the strap assembly 310. Generally, electrical pathways 322A and 322B extend to a point along the strap assembly 310 that is in close proximity to the target area on the user’s face. Thus, the electrical pathways 322A and 322B generally have a first end positioned at the electrical contacts 320A, 320B, respectively, and a second end position at some other portion of the strap assembly 310 near the target area of the user.

When the end 317A of lower strap 316A is brought near lateral strip 364A, magnets 319A and 319B are magnetically attracted to magnets 365A and 365B. The magnetic attraction secures end 317A of lower strap 316A to lateral strip 364A, which causes electrical contact 371A to physically contact electrical contact 320A and electrical contact 371B to physically contact electrical contact 320B. Electrical pathway 369A is thus electrically connected to electrical pathway 322A, while electrical pathway 369B is electrically connected to electrical pathway 322B. Because of the electrical connection between lateral strip 364A, the annular projection 358 of the frame 350, and the connector 370, electrical pathways 322A, 322B (which extend into the strap assembly 310) are electrically connected to the connector 370. Thus, the frame 350 can be physically and electrically connected to the strap assembly 310.

The end 317A of lower strap 316A includes a rotation-locking feature, and the lateral strip 364A includes a corresponding rotation-locking feature. In the illustrated implementation, rotation-locking feature of end 317A of the lower strap 316A is a T-shaped projection 324 that extends away from magnets 319A and 319B, and the rotation-locking feature of the lateral strip 364A is a channel 373 defined between magnets 365A and 365B sized to receive at least a portion of the T-shaped projection 324. Generally, the linear portion of the T-shaped projection 324 can fit into the channel 373 when the end 317A of lower strap 316A is secured to lateral strip 364A. The T-shaped projection 324 is thus locked between the magnets 365A and 365B, preventing magnets 365A and 365B from rotating relative to magnets 319A and 319B. This locked rotation turn ensures that electrical contact 371A remains physically touching electrical contact 320A, and that electrical contact 371B remains physically touching electrical contact 320B. Additionally, the lower curved portion of the T-shaped projection 324 generally fit underneath magnets 365A and 365B (relative to the plane of FIG. 7A), which prevents the lower strap 316A from inadvertently being pulled away from lateral strip 364A.

The electrical pathways 322A and 322B that extend from end 317A of lower strap 316A into the strap assembly 310 can be formed in a variety of different manners. In some implementations, the electrical pathways 322A and 322B are formed from wires that run through a generally hollow interior of the lower strap 316A and/or any other portion of the strap assembly 310. In other implementations, the strap assembly 310 is not hollow, and the wires forming the electrical pathways 322A and 322B are instead woven in with the material forming the strap assembly 310. In still other implementations, electrical pathways 322A and 322B are formed by electrical traces that run along the surface of the lower strap 316A and the rest of the strap assembly 310.

By utilizing the electrical pathways and electrical contacts of the user interface 300, the one or more sensors 390 can be placed at any suitable location, and can be electrically connected to the connector 370. By coupling the connector 370 with a conduit having its own electrical pathways (e.g., wires or traces inside the conduit), the one or more sensors 390 can be electrically coupled to a control system and memory device disposed in or near the respiratory therapy device.

In some implementations, the one or more sensors 390 are positioned near the connector 370. In this implementation, the one or more sensors 390 are electrically connected to one or more of the electrical contacts 378A-378D of the connector 370, so that data generated by the one or more sensors 390 can be transmitted via electrical contacts 378A-378D. In these implementations, the one or more sensors 390 may be positioned inside the connector 370.

In other implementations, the one or more sensors 390 are positioned near the frame 350. For example, the one or more sensors 390 can be positioned between the user’s face and the cushion 330, between the cushion 330 and the frame 350, or inside the annular aperture 356 of the frame 350. In this implementation, the one or more sensors 390 are electrically connected to one or more of the electrical contacts 368A-368D of the frame 350, so that data generated by the one or more sensors 390 can be transmitted via electrical contacts 368A-368D of the frame 350, and electrical contacts 378A-378D of the connector 370.

In further implementations, the one or more sensors 390 are positioned near either of the strap attachments points of the frame 350. In some of these implementations, the one or more sensors 390 are positioned in or near the extension strip 360, and is electrically connected through the extension strip 360 to the frame 350 and the connector 370. In others of these implementations, the one or more sensors 390 can be positioned, for example, near the magnet 366A of the lateral strip 364A, and electrically connected to one or both of electrical contacts 371A and 371B, so that data generated by the one or more sensors 390 can be transmitted through electrical contacts 371A and 371B of the lateral strip 364A, electrical contacts 368A-368D of the frame 350, and electrical contacts 378A-378D of the connector 370.

In still other implementations, the one or more sensors 390 are positioned near the end of one of the lower straps, such as near end 317A of lower strap 316A. The one or more sensors 390 can be electrically connected to one or both of electrical contacts 320A and 320B, so that data generated by the one or more sensors 390 can be transmitted through electrical contacts 320A and 320B, electrical contacts 371A and 371B of the lateral strip 364A, electrical contacts 368A-368D of the frame 350, and electrical contacts 378A-378D of the connector 370.

In some implementations, the one or more sensors 390 are positioned along the strap assembly 310 adjacent to the target area of the user. In these implementations, the one or more sensors 390 can be electrically connected to the electrical pathway extending through the strap assembly 310, such as electrical pathways 322A and 322B. Thus, data generated by the one or more sensors 390 can be transmitted through electrical pathways 322A and 322B, electrical contacts 320A and 320B, electrical contacts 371A and 371B of the lateral strip 364A, electrical contacts 368A-368D of the frame 350, and electrical contacts 378A-378D of the connector 370. Further, the one or more sensors 390 can include contact portions that contact the target area of the user, and a wire that electrically connects the contact portion of the sensor with the electrical pathways in the strap assembly 310, such as electrical pathways 322A and 322B.

In even further implementations, instead of being electrically connected to the control system and memory device through a conduit, the one or more sensors 390 can be electrically connected to a processing device (such as a microprocessor) that is located in the connector 370. In these implementations, the microprocessor is electrically connected to the electrical contacts 378A-378D of the connector 370, so that data generated by the sensor can be transmitted via the strap assembly 310, the frame 350, and the connector 370 to the microprocessor.

The user interface 124 and/or the conduit 126 may also include one or more safety features to mitigate the risk of electrical shock due to excessive leakage currents, which may result from worn or defective circuity, or inadvertently exposed components. In some implementations, opto-isolators or 1:1 transformers can be used to electrically isolate various components. In addition, heating of any of the electrical components can be mitigated, for example using a variety of different insulators.

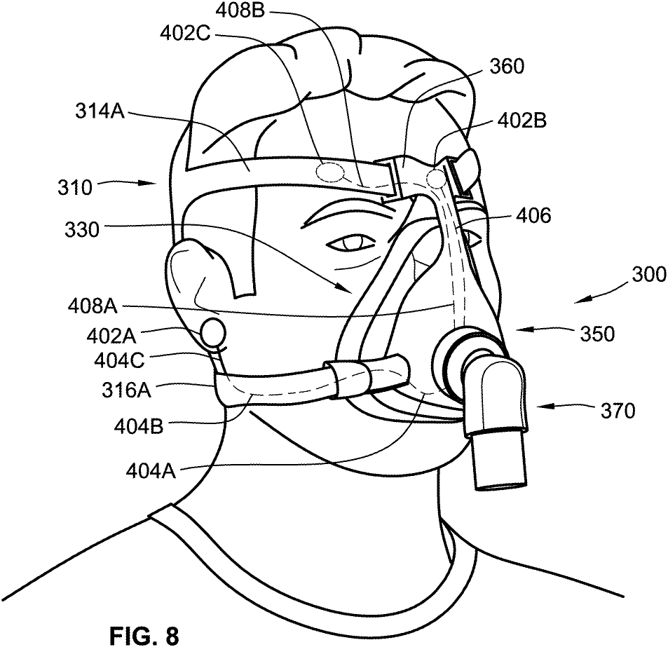

FIG. 8 illustrates a user (such as user 210) wearing the user interface 300 with three different sensors coupled to the strap assembly 310 and being positioned adjacent to or abutting different portions of the user. As shown, the strap assembly 310 is positioned around the user’s head, and is coupled to the frame 350. The cushion 330 is attached to the frame 350 and positioned between the user’s face and the frame 350. The connector 370 is coupled to the frame 350.

The user interface 300 in FIG. 8 includes three sensors 402A, 402AB, and 402C located at or adjacent to different areas of the strap assembly 310, and abutting different areas on the user. Sensor 402A is located adjacent to the lower strap 316A, sensor 402B is located in the extension strip 360, and sensor 402C is located in the upper strap 314A.

In the illustrated implementation, sensor 402A is clipped to the user’s ear, and can be an SpO₂ sensor used to measure peripheral oxygen saturation. By clipping the SpO₂ sensor to the user’s ear instead of another portion of the user (such as a finger or a toe), more reliable measurements of peripheral oxygen saturation can be obtained. Sensor 402A is electrically connected to the connector 370 through the frame 350, a first electrical pathway 404A, a second electrical pathway 404B, and a third electrical pathway 404C. The first electrical pathway 404A is disposed in the frame 350, and can be a wire or an electrical trace. The first electrical pathway 404A out to a strap attachment point of the frame 350, where the lower strap 316A is coupled to the frame 350. The second electrical pathway 404B extends through the lower strap 316A itself, and can be a wire or an electrical trace positioned inside the lower strap 316A or on the surface of lower strap 316A. The first electrical pathway 404A and the second electrical pathway 404B can be electrically coupled using magnets located in the frame 350 and the lower strap 316A, as illustrated in FIG. 7. The third electrical pathway 404A extends out of the lower strap 316A to the sensor 402A clipped to the user’s ear. The third electrical pathway 404A is thus generally formed as a wire. Thus, data generated by the sensor 402A can be transmitted via the lower strap 316A, the frame 350, and the connector 370.

In other implementations, sensor 402A could be located adjacent to the neck or throat of the user. In these implementations, the second electrical pathway 404B can extend out of the lower strap 316A and downward to the sensor 402A.

In the illustrated implementation, sensor 402B is a contact sensor that abuts the user’s forehead (such as an EEG sensor) when the user interface 300 is worn by the user. The sensor 402B can measure brain activity at of the frontal lobe, which can aid in determining which stage of sleep the user, and in detecting arousals and micro-arousals during the user’s sleep session. The sensor 402B is electrically connected to the connector 370 through the frame 350 and through electrical pathway 406. Electrical pathway 406 generally extends from the frame 350 and up to the extension strip 360, and can be a wire or an electrical trace. Generally, sensor 402B is positioned outside of the extension strip 360 between the extension strip 360 and the user’s forehead. Sensor 402B can be electrically connected to the electrical pathway 404 at the backside surface of the extension strip 360, or the electrical pathway 404 may protrude slightly from the backside surface (e.g., as a wire) to electrically connect with the sensor 402B. Thus, data generated by the sensor 402B can be transmitted via the extension strip 360, the frame 350, and the connector 370.

In the illustrated implementation, sensor 402C is a contact sensor that contacts the user’s temple (such as an EOG sensor) when the user interface 300 is worn by the user. Sensor 402C is electrically connected to the connector 370 through the frame 350, a first electrical pathway 408A, and a second electrical pathway 408B. The first electrical pathway 408A can generally be the same as or similar to electrical pathway 406, and thus extends from the frame 350 up to the extension strip 360. However, the first electrical pathway 408A is connected to the second electrical pathway 408B, which extends through the upper strap 314A. In some implementations, the transition between the first electrical pathway and the second electrical pathway 408B can utilize magnets, as illustrated in FIG. 8. In other implementations, the upper strap 314A is looped through an aperture in the extension strip 360, and magnets are not used. In these implementations, the first electrical pathway 408A may end in a wire extending from the extension strip 360 toward the upper strap 314A. The wire may then extend into the upper strap 314A, thus beginning the second electrical pathway 408B.

The second electrical pathway 408B extends toward the user’s temple, where it electrically connects with the sensor 402C. Similar to sensor 402B, sensor 402C can be positioned between the user’s temple and the upper strap 316A. Sensor 402C can be electrically connected to the second electrical pathway 408B at the backside surface of the upper strap 316A, or the second electrical pathway 408B may protrude slightly from the backside surface (e.g., as a wire) to electrically connect with the sensor 402C. Thus, data generated by the sensor 402C can be transmitted via the upper strap 316A, the extension strip 360, the frame 350, and the connector 370.

The system 100 can also include sensors configured to determine if the user is sleeping on their back or on either side. In some implementations, sensors can be placed in the user interface 124 or the conduit 126 that measure relative airflow between different sides of the conduit 126. If the user is sleeping on their side, one of the sensors will measure less airflow relative to the other side, which enables the system 100 to determine which side the user is sleeping on. If the air between the sensors is generally equal, the system 100 can determine that the user is sleeping on their back. This information can, in some examples, be used to provide an estimate of the integrity or wear and tear on the mask.

In some implementations, existing electrical wires that may be inside the conduit can be used with user interface 300. For example, the conduit may include two wires coupled to a thermistor, which can be used as a temperature sensor. The thermistor can be removed, and these two wires can be electrically connected to the connector, in order to transmit data from the one or more sensors 390. In another example, the thermistor is retained but the connector is configured to bypass the thermistor and electrically connect to the two wires. In yet another example, the conduit may include wires used to heat air flowing through the conduit. These wires can be used instead as a voltage source (for example by attaching a voltage regulator component such as a Zener diode) to power the one or more sensors 390 or any other sensors or components in the user interface 300 that require power to operate.

In some implementations, the airflow through the conduit and the connector 370 can be used to power the one or more sensors 390 and any other components. In these implementations, a small power generator can be placed in the conduit or the connector 370, in the path of the pressurized air flowing through the conduit and the connector 370. The air flowing through and past the power generator can be used to generate some or all of the required power. In some of these implementations, the power generator includes a turbine that spins as the air flows through the conduit and connector 370, to thereby generate power. Other implementations can include a thermoelectric generator that converts heat flux to electricity. The power generator can include nanomaterials.

The one or more sensors 390 (which can generally include one or more of the sensors 130, or other sensors) can be used for a variety of different purposes. In one implementation, the one or more sensors 390 are used to detect mouth leak (e.g., pressurized air entering the noise and exiting through the mouth without entering the user’s throat, trachea, or lungs). In this implementation, sensors located in the cushion 330 and/or in the frame 350 can be used to detect air leaking from the user’s mouth. These sensors could include a pressure sensor (such as pressure sensor 132), a flow rate sensor (such as flow rate sensor 134), a CO₂ sensor, an O₂ sensor, an acoustic sensor, a microphone, or any other combination of sensors.

Generally, many respiratory therapy devices that can be used to provide respiratory treatment to a user during a sleep session contain their own sensors to measure various parameters. However, user interface 300 can be used on conjunction with a respiratory therapy device that does not contain any separate sensors. In these implementations, the respiratory therapy device includes a housing defining an inlet and an outlet, and has a blower motor within the housing that is in fluid communication with the inlet and the outlet. The respiratory therapy device also includes a control system with one or more processors that execute machine-readable instructions stored on a memory device to cause the blower motor to flow pressurized air out of the outlet. However, because any required sensors can be placed in the user interface 300, the respiratory therapy device does not include its own sensors.

For example, pressure sensors and flow rate sensors are often used in respiratory therapy devices to monitor operation of the blower motor and the amount of air that is being delivered to the user. Because the user interface 300 can include a pressure sensor and a flow rate sensor, the respiratory therapy device does not need its own pressure sensor and flow rate sensor. The pressure sensor and the flow rate sensor of the user interface 300 can generate data related to the respiratory therapy device and/or the user of the respiratory therapy device, and that data can be transmitted via the user interface 300 and a conduit fluidly connecting the user interface 300 and the respiratory therapy device. The control system of the respiratory therapy device can use the data from the pressure sensor and the flow rate sensor to operate the blower motor.

Generally, any of the above techniques or features for electrically connecting components can be used in other locations on the user interface 300. For example, the strap assembly 310 could have only straps that couple to the frame 350 using magnets. In another example, the strap assembly 310 could have only straps that couple to the frame 350 by looping through apertures in the frame 350 and the extension strip 360. In still other example, the lower straps 316A, 316B could loop through apertures in the frame 350, while the upper strap 314A, 314B couple to the extension strip 360 using magnets. In still other example, the frame 350 may not have the extension strip 360, and thus the upper straps 314A, 314B are coupled to the body 352 of the frame 350, closer to the lower straps 316A, 316B.

Further, the user interface 300 is not limited to the specific number or arrangement of electrical contacts in the connector 370, the frame 350, or the strap assembly 310 as is illustrated. The user interface 300 can generally include any arrangement of electrical contacts and electrical pathways through the various components, in order to place the one or more sensors 390 in their desired locations while also electrically connecting each of the one or more sensors 390 back to the connector 370. For example, the frame 350 and connector 370 could each include single electrical contacts for a single sensor, multiple sets of electrical contacts for a single sensor, more or less than four electrical contacts for any number of sensors, etc. Finally, any of the one or more sensors 390 can be located in any suitable location in the strap assembly 310 or in other portions of the user interface 300.

In other implementations, the various electrical pathways are not formed by wires or by traces on or in the various parts of the user interface 124 or the conduit 126, but instead are wireless electrical pathways or inductive electrical pathways. Wireless electrical pathways can use energy harvesting and wireless communication. Inductive electrical pathways can utilize magnetic fields and/or electrical fields.

In some implementations, the strap assembly 310 includes hollow tubes that extend around the user 210’s face. The hollow tubes can generally have all of the same characteristics as the upper and lower straps 314A, 314B, 316A, 316B, except that they are hollow along their entire length. Any wires or sensors can then be positioned within the hollow tubes that make up the strap assembly 310.

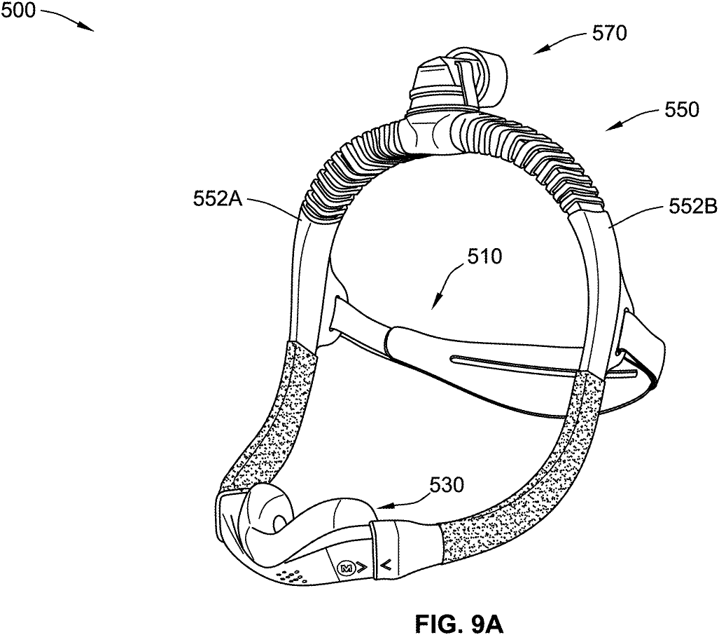

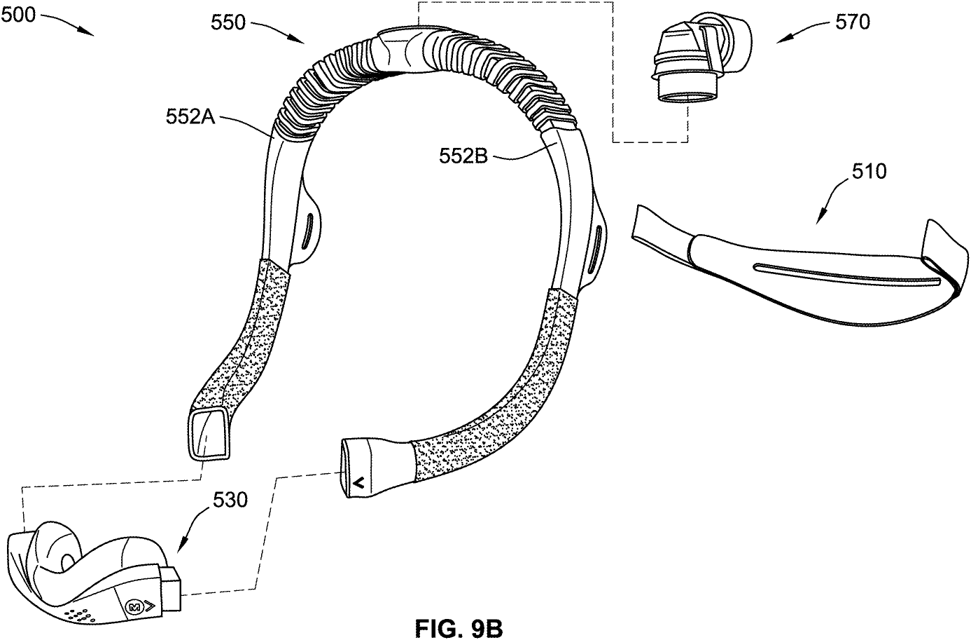

FIGS. 9A and 9B illustrate a perspective view and an exploded view, respectively, of a user interface 500 that can include a variety of different sensors according to aspects of the present disclosure. The user interface 500 includes a strap assembly 510, cushion 530, a frame 550, and a connector 570. The strap assembly 510 can be coupled to the frame 550, and when the user dons the user interface 500, the strap assembly 510 is be positioned generally about the back of the user’s head, such that the user’s head is positioned between the strap assembly 510 and the frame 550. The cushion 530 can be attached to lower ends of the frame 550 so that the cushion 530 is positioned near the user’s face when the user dons the user interface 500, so that the cushion 530 forms a seal on the user’s face. The connector 570 is configured to be inserted into an aperture in the frame 550, to thereby couple the connector 570 to the frame 550. The conduit 126 of the respiratory therapy system 120 can be coupled to the other end of the connector 570, to thereby connect the respiratory therapy system 120 to the user interface 500. In other implementations, the connector 570 can be optional and the frame 550 can alternatively connect directly to conduit of the respiratory therapy system.

The user interface 500 is configured to deliver pressurized air from the conduit 126 of the respiratory therapy system 120 to the user through the cushion 530 and the frame 550, or more specifically, to the volume of space around the mouth and/or nose of the user and enclosed by the cushion 530. In the illustrated implementation, the user interface 500 includes hollow portions 552A and 552B to provide two passageways for the pressurized air that fluidly connect the cushion 530 to the connector 570. In this manner, the cushion 530 is in fluid communication with the interior of the connector 570. When the user dons the user interface 500, the hollow portions 552A and 552B will generally be positioned on either side of the user’s head/face. In other implementations, the user interface 500 may only include one of the hollow portions 552A and 552B to provide a single passageway for the pressurized air, with the other portion being a solid portion that does not form a passageway for the pressurized air. In still other implementations, both portions 552A and 552B can be solid, and the frame 550 may one or more tubes (or other hollow portions) that form one or more passageways for the pressurized air between the connector 570 and the user’s mouth and/or nose. Thus, in the implementation of FIGS. 9A and 9B, the conduit 126 of the respiratory therapy system 120 is generally attached to the frame of the user interface at the top of the user’s head, instead of in front of the user’s face.

The user interface 500 can include a variety of different electrical pathways, similar to user interface 500. For example, the connector 570 can be similar to the connector 370, and include electrical contacts on the end of the connector 370 that are configured to mate with the conduit 126 of the respiratory therapy system 120. The connector 570 can also include annular electrical contacts at the opposite end of the connector 370 that are configured to mate with the frame 550. The frame 550 in turn can be similar to the frame 350, and include electrical contacts near the end of the frame 550 that mate with connector 570. Thus, the electrical contacts in the frame 550 and the connector 570 allow an electrical connection to be made between the conduit 126 of the respiratory therapy system 120 and the frame 550. Electrical pathways can then be formed from the frame 550 to a target area for a sensor, through any desirable path. For example, wires or traces can extend from the frame 550 to the user’s face; from the frame 550, through the strap assembly 510, and to the user’s face; from the frame 550, through the cushion 530, and to the user’s face; from the frame 550, through the strap assembly 510 and the cushion 530, and to the user’s facer; or from the frame 550, through the cushion 530 and the strap assembly 510, and to the user’s face. In this manner, the frame 550 can be physically and electrically connected to the strap assembly 510, and the connector 570 can be physically and electrically connected to the frame 550. Similar to use interface 300, sensors can be positioned in generally any target area on the user or around the user, and electrical connections can be formed to the sensors using any of the components of the user interface 500.

The one or more sensors 390 of the user interface 300 or of the user interface 500 can include a variety of different sensors in different locations to accomplish a variety of different sensing tasks. In some implementations, the one or more sensors 390 includes one or more EEG sensors that contact a portion of the user’s head, which could include the user’s forehead and/or scalp. The EEG sensors measure electrical activity associated with the user’s brain (e.g., brain activity), and can be used to detect sleep stages and/or to detect micro sleep arousals. The EEG sensors could also be implemented in an earbud positioned in the user’s ear, which can additionally be used to monitor sound and temperature. The one or more sensors 390 can include multiple EEG sensors contacting a variety of different areas on the user’s scalp, which can then be used for quantitative EEG, also referred to as brain mapping.

In some implementations, the one or more sensors 390 includes one or more ECG sensors configured to measure electrical activity of the user’s heart (e.g., cardiac activity). The ECG sensors can measure the difference in electrical activity between different portions of the user’s, such as between different portions of the user’s head, between the user’s ears, between the user’s chin and one of the user’s ear, etc.

In some implementations, the one or more sensors 390 includes one or more EOG sensors configured to measure movements of the user’s eyes. The EOG sensors can thus be used to detect when the user is moving their eyes, which in turn can aid in determining when the user is in a REM sleep stage.

In some implementations, the one or more sensors 390 includes one or more EMG sensors configured to measure electrical activity of the user’s muscles. The EMG sensors can be placed near muscles in the user’s face to detect facial movements. For example, the EMG sensors can be placed near the user’s jaws to detect jaw movement, which can be indicative of the user grinding their teeth during a sleep session, also known as bruxism. Jaw movement detected by the EMG sensors (and/or other muscle activity) can also be used to aid in determining whether the user is experiencing a seizure.

In some implementations, the one or more sensors 390 includes one or more microphones that can be used to detect a variety of different sounds, such as breathing sounds (e.g. mouth or nose breathing), noises from the user interface (which can occur if the user interface moves during the sleep session, such as when the user moves), background noises, noises caused by air leaking from the user interface, etc. The microphones can also be used to determine if any detected air leaks are intentional and due to the operation of any vents in the user interface, or if the detected air leaks are unintentional and due to a poor seal between the user and the user interface. A breathing signal can be derived from the microphone data, which can indicate the quality of the user’s breathing (e.g., normal, slow, fast, raspy, wheezing, whistling, etc.). In some implementations, the microphone can be implemented as an earbud positioned in or near the user’s ear, which could also be used as an EEG sensor and a temperature sensor.

In some implementations, the one or more sensors 390 includes one SpO₂ sensors configured to measure the user’s peripheral oxygen saturation. The SpO₂ sensors can be placed in a variety of locations, including near the user’s ears, nose, lips, and/or forehead. The SpO₂ sensors can be reflective sensors or transmissive sensors, and can utilize, in some implementations, green LEDs and/or red LEDs.

In some implementations, the one or more sensors 390 includes one or more GSR sensors configured to measure electrical properties of the user’s skin (also referred to as electrodermal activity, or EDA), The GSR sensors can be located on the user’s face, and can aid in determining the user’s emotions, performing lie detection, and performing sleep analysis.

In some implementations, the one or more sensors 390 includes one or more motion sensors, which can include accelerometers, gyroscopes, magnetometers, inertial measurement units (IMUs), or any combination thereof. The motion sensors can be used to measure activity (such as movement during the sleep session), the user’s gait if walking, fall detection (for example if the user is elderly and at risk of falling out of bed or falling when walking), etc. The motion sensors can be used to measure movements of the user due to the user breathing (e.g., the user’s chest rising and falling during respiration), which can in turn be used to derive a breathing signal. The motion sensors can measure the rate of movement to determine the breathing rate; can detect the user’s chest struggling to move during breathing which can be indicative of an obstructive sleep apnea; and can detect when the chest is not moving at all due to a central sleep apnea where the user’s brain does not signal to breathe. The breathing signal can indicate the quality of the user’s breathing (e.g., normal, slow, fast, raspy, wheezing, whistling, etc.). In some implementations, the motion sensors can be used to determine if there is any movement of the user interface on the user’s head, which can indicate that the user interface does not fit properly. This determination can also be based on data from tension sensors, which can represent the tension in the straps of the user interface, and whether the user interface is tightened properly on the user’s head. In some implementations, the motion sensors can be used to determine the user’s position in bed, which can aid in determining whether the user interface is improperly fitted and causing leaks or poor air flow.

In some implementations, the one or more sensors 390 includes one or more analyte sensors that can be used to detect analytes in the user’s breath, such as ketones. The analyte sensors can thus be used to perform breath sampling and analysis. The analyte sensors can also detect analytes in the air, and thus can be used to perform air quality analysis.

In some implementations, the one or more sensors 390 includes one or more pressure sensors that can be used to determine the pressure of the pressurized air delivered to the user’s airway. These pressure sensors can be placed in the user interface closer to the user’s mouth and/or nose than pressure sensors in the conduit 126 or in the respiratory therapy device 122, and thus can in some implementations provide a more accurate measure of the pressure of the pressurize air.

In some implementations, the one or more sensors 390 includes one or more RF sensors, one or more sonar sensors, one or more flow sensors (which can be in addition to or as an alternative to any flow sensors in the respiratory therapy system 120), one or more temperature sensors (which can be used to measure the user’s core temperature at the user’s temples or in the user’s ears, or the temperature of the user interface), one or more heart rate sensors (which can be used to measure the user’s heart rate, for example at the user’s temples), and others. The temperature sensor can be implemented as an earbud positioned in or near the user’s ear, which could also be used as an EEG sensor and a microphone. The heart rate sensors can include PPG sensors, RF sensors, or even motion sensors that are able to detect motion caused the user’s heartbeat (such as movement of the user’s chest or movement due to a pulse in a vein or artery).

The one or more sensors can be used for a variety of different applications. In some implementations, the one or more sensors 390 can be used to perform polysomnography (PSG), which measures a variety of body functions while the user is asleep. PSG can use EEG sensors to measure brain activity, ECG sensors to measure cardiac activity, EOG sensors to measure eve movements, EMG sensors to measure muscle activity, and other sensors. PSG is commonly conducted during sleep studies, and thus aspects of the present disclosure allow a PSG to be conducted using a user interface that the user will already be wearing during their sleep session. Because of the electrical pathways that can be formed in the user interface that is already being worn by the user, the sensors required to perform PSG can be attached and/or positioned near the patient as needed through the user interface.

In some implementations, the one or more sensors 390 can be used for emotion mapping. The one or more sensors 390 can detect a variety of different characteristics, including facial expressions and body positions, that may be relevant to the user’s emotional state. The one or more sensors 390 can also be used to detect spontaneous emotions versus forced emotions. The user’s heart rate and breathing rate detected by the one or more sensors 390 can also be used to determine the user’s emotional state, as they can be indicative of the user’s stress levels. Speech detected by the one or more sensors 390 can also be used to aid in determining the user’s emotional state. Data from galvanic skin response sensors can also aid in determining the user’s emotional state.

The data from the one or more sensors 390 can be used to test for conditions other than the sleep-related condition that the user uses the respiratory therapy system 120 to treat. For example, the data can be used to determine if the user had any underlying conditions such as atrial fibrillation, which may be evidenced by intermittent cardiac abnormalities, breathing abnormalities, etc. The data from the one or more sensors 390 can also be used to determine the level of the user’s cognitive functioning, including checking for signs of early onset Alzheimer’s, dementia, and other cognitive abnormalities. The data from the one or more sensors 390 can also be used to determine the user’s level of drowsiness, which can be connected to conditions such as the cold or flu, or other chronic diseases. In some implementations, the data from the one or more sensors 390 can be used to detect any discomfort or pain being experienced by the user, and to determine potential causes of the pain/discomfort (e.g., a specific body or neck position may be painful to the user during the sleep session). In some implementations, the one or more sensors 390 can be used to detect various characteristics of the user’s bedroom (or any other room that the user may be in during the sleep session). For example, a sonar sensor could be used to identify and map physical features of the room. In some implementations, the data from the one or more sensors 390 can be used to provide feedback to the user after their sleep session. The feedback can include providing the user with the data itself, and/or analysis based on the data. By using the one or more sensors 390 to detect and monitor these other conditions, the user interface 300 and/or the user interface 500 provide a more efficient mechanism for detecting and monitoring other conditions in users who suffer from these other conditions, and/or require other therapies to treat these other conditions.

In some implementations, the user interface may include one or more actuators configured to perform functions based on data from the one or more sensors 390. The actuators can be used to adjust the fit of the user interface on the user (for example by tightening or loosening the strap assembly, or by re-positioning the user interface relative to the user’s face), to wake up the user during the sleep session, or to perform any other desired function.

In some implementations, the user interface may include components to power the one or more sensors 390 separate from any power provided by the respiratory therapy system 120. The user interface can also include one or more communication interfaces (e.g., transmitters, receivers, transceivers, data ports, etc.) that allow the data generated by the one or more sensors 390 to be transferred and stored independently from the respiratory therapy system 120. Thus, the user interface can in some implementations form an independent sensor suite that is able to independently generate and transfer data.

One or more elements or aspects or steps, or any portion(s) thereof, from one or more of any of claims 1-69 below can be combined with one or more elements or aspects or steps, or any portion(s) thereof, from one or more of any of the other claims 1-69 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A user interface of a respiratory therapy system, the user interface comprising:
a strap assembly configured to be positioned generally about at least a portion of a head of a user when the user interface is worn by the user;
a frame coupled to the strap assembly, the frame defining an aperture;
a cushion coupled to the frame;
a connector having a first portion and a second portion, the first portion being configured to be at least partially coupled to the aperture of the frame such that the connector is physically and electrically connected to the frame; and
one or more sensors coupled to the user interface, the one or more sensors including at least one sensor coupled to the connector
wherein data generated by the at least one sensor is transmitted via an electrical pathway between the at least one sensor and the connector.

2. (canceled)

3. The user interface of claim 1, wherein the at least one sensor is electrically connected to the frame and to the connector such that data generated by the at least one sensor can be transmitted via the electrical pathway.

4-7. (canceled)

8. The user interface of claim 1, wherein the respiratory therapy system includes a respiratory therapy device, a conduit, and the user interface, the conduit configured to be in fluid communication with the respiratory therapy device and the user interface, the second portion of the connector configured to be physically and electrically coupled to the conduit, such that the connector is in fluid communication with the conduit.

9. (canceled)

10. The user interface of claim 8, wherein the conduit is configured to be further electrically connected to a control system, such that data generated by the at least one sensor can be transmitted to the control system via the connector and the conduit, the control system being configured to modify operation of the respiratory therapy device based at least in part on the data generated by the at least one sensor.

11-16. (canceled)

17. The user interface of claim 1, wherein the connector includes one or more electrical contacts, and wherein the at least one sensor is electrically connected to at least one of the one or more electrical contacts of the connector to thereby electrically connect the at least one sensor to the connector, such that data generated by the at least one sensor can be transmitted via the electrical contacts of the connector.

18. The user interface of claim 15, wherein the frame includes one or more electrical contacts.

19. (canceled)

20. The user interface of claim 18, wherein each of the one or more electrical contacts of the connector contacts a corresponding one of the one or more electrical contacts of the frame when the connector is coupled to the frame, to thereby electrically connect the frame to the connector.

21-24. (canceled)

25. The user interface of claim 20, wherein the at least one sensor is electrically connected to at least one of the one or more electrical contacts of the frame, such that data generated by the at least one sensor can be transmitted via the one or more electrical contacts of the frame and the one or more electrical contacts of the connector.

26-69. (canceled)

70. The user interface of claim 1, wherein the one or more sensors further includes an additional sensor coupled to the strap assembly, the cushion or the frame, and wherein data generated by the additional sensor coupled to the strap assembly, the cushion, or the frame is transmitted to the at least one sensor coupled to the connector via an electrical pathway between the additional sensor and the connector.

71. The user interface of claim 70, wherein the additional sensor coupled to the strap assembly, the cushion, or the frame is a radio-frequency (RF) transmitter configured to transmit data, and wherein the at least one sensor coupled to the connector is an RF receiver configured to receive the data transmitted by the RF transmitter.

72. The user interface of claim 70, wherein the additional sensor is coupled to the cushion and is electrically connected to the frame via one or more wires electrically connected to the additional sensor and extending into the frame.

73. The user interface of claim 72, wherein the electrical pathway between the additional sensor and the connector includes an electrical connection between the frame and the connector.

74. The user interface of claim 70, wherein the at least one sensor coupled to the connector and the additional sensor coupled to the strap assembly, the cushion, or the frame are both non-contact sensors.

75. The user interface of claim 1, further comprising a first hollow portion and a second hollow portion coupled to the cushion and the connector, the first hollow portion and the second hollow portion each being a passageway that fluidly connects the cushion to the connector.

76. The user interface of claim 75, wherein the first hollow portion and the second hollow portion are formed as part of the frame of the user interface, and extend away from the cushion and towards the aperture of the frame.

77. The user interface of claim 74, wherein the one or more sensors further includes an additional sensor coupled to the cushion and electrically connected to the frame via one or more wires electrically connected to the additional sensor and extending into the frame, and wherein the one or more wires electrically connected to the additional sensor include (i) at least one wire extending through or along the first hollow portion toward the aperture of the frame, (ii) at least one wire extending through or along the second hollow portion to the aperture of the frame, or (iii) both (i) and (ii).

78. A user interface of a respiratory therapy system, the user interface comprising:
a strap assembly configured to be positioned generally about at least a portion of a head of a user when the user interface is worn by the user;
a frame physically connected to the strap assembly, the frame defining an aperture;
a cushion coupled to the frame;
a connector configured to be at least partially positioned within the aperture of the frame
such that the connector is physically and electrically connected to the frame; and a first sensor coupled to the connector; and
a second sensor coupled to the cushion,
wherein the first sensor is electrically connected to the second sensor via an electrical pathway between the frame and the connector.

79. The user interface of claim 79, wherein the first sensor is a radio-frequency (RF) receiver and the second sensor is an RF transmitter.

80. The user interface of claim 79, wherein the electrical pathway between the frame and the connector is at least partially a wireless electrical pathway, at least partially an inductive electrical pathway, or both.

81. The user interface of claim 79, wherein the electrical pathway between the frame and the connector includes at least one wire extending at least partially through the frame.
